(19) United States
(12) Patent Application Publication
(10) Pub. No.: US 2023/0089803 A1
(43) Pub. Date: Mar. 23, 2023

(54) ACCURACY IN BLOOD GLOCUSE MEASUREMENT USING PREDICTIVE THERMAL MODELING

(71) Applicant: TELADOC HEALTH, INC., Purchase, NY (US)

(72) Inventors: Douglas Yuk, Fremont, CA (US); Randy Buswell, Fremont, CA (US); Gene V. Kozin, San Jose, CA (US); Yongbo Wang, Redondo Beach, CA (US); Frederick Barrigar, Los Altos, CA (US)

(21) Appl. No.: 17/734,970

(22) Filed: May 2, 2022

Related U.S. Application Data
(60) Provisional application No. 63/201,472, filed on Apr. 30, 2021.

(51) Int. Cl. B01L 3/00; G01K 1/02; G01K 7/22; C12Q 1/26

(52) U.S. Cl. CPC B01L 3/5023; G01K 1/026; G01K 7/22; C12Q 1/26; B01L 2300/0663; B01L 2300/0645; B01L 2300/027; B01L 2300/0825; B01L 2300/069

(57) ABSTRACT

System, method, and computer program product for improving the accuracy of blood glucose measurements in a blood glucose meter by compensating for thermal effects of the glucose meter hardware. A machine learning model is used to predict the reaction site temperature on a blood glucose test strip based on one or more temperature measurements and device status information. The model receives several inputs including at least one temperature measurement sensed near the reaction site as well as device usage and other factors that may influence the reaction site temperature. The model provides a predicted reaction site temperature to the glucometer software or firmware to be used in a blood glucose calculation.

1.

### CROSS-REFERENCE TO RELATED APPLICATIONS

This application claims priority to U.S. provisional application No. 63/201,472, filed Apr. 30, 2021, the contents of which are hereby incorporated by reference in their entirety.

### BACKGROUND

One method of blood glucose measurement employs a test strip designed to receive a sample of blood and expose a portion of the blood sample to an enzyme known as glucose oxidase. The enzyme removes electrons from glucose molecules in the blood. A mediator then passes these electrons into an electric circuit embedded in the test strip, which generates an electric current proportional to the amount of glucose in the blood sample. A blood glucose meter, or glucometer, connected to the test strip can measure this current to calculate the amount of glucose in the blood sample. The enzymatic reaction is temperature dependent and, thus, so are the resultant electric current and glucose measurement. Because the glucose measurement is temperature dependent, to ensure an accurate measurement, the temperature at the enzymatic reaction site must be known. A temperature sensor built into the blood glucose meter itself can provide some indication of the temperature of the meter itself, but this may not correspond closely enough with the temperature of the test strip to provide an accurate blood glucose reading. Some prior art test strips include a built-in temperature sensor that can be read by the glucose meter when the strip is inserted into the meter. This solution increases the complexity of the test strip and, therefore, increases its cost.

### SUMMARY

It would be advantageous to provide a system and method in a blood glucose meter for accurately sensing the temperature at the reaction site of a blood glucose test strip without the added cost of building a temperature sensor into the test strip itself. Moreover, it would be advantageous to provide a system and method in a blood glucose meter to more accurately measure the temperature of the test strip using one or more temperature sensors built into the glucose meter itself.

To achieve these and other advantages, one aspect of the disclosure is a blood glucose meter. The blood glucose meter includes a test strip interface configured to be electrically coupled to a test strip that contains a blood sample. The meter also includes a first temperature sensor located a first distance from the test strip interface, a user interface, and a first processor. The first processor is coupled to the test strip interface, the first temperature sensor, and the user interface. The first processor is configured to calculate a glucose value associated with the blood sample based on a plurality of inputs. The inputs include an electrical characteristic of the test strip, a first temperature value received from the first temperature sensor, and a device status indicator.

A second aspect of the disclosure is a method for calculating a blood glucose level. The method includes obtaining, at a first processor, a measurement of an electrical characteristic of a blood glucose test strip from test strip interface of a blood glucose meter. The method further includes obtaining, at the first processor, a first temperature value from a first temperature sensor located a first distance from the test strip interface. The method further includes obtaining, at the first processor, at least one device status indicator. The method includes calculating a blood glucose measurement based on a plurality of inputs including the measured electrical characteristic, the first temperature value, and at least one status indicator, wherein each of the plurality of inputs is weighted in accordance with a model. In addition, the method includes displaying the calculated blood glucose measurement on a user interface coupled to the first processor.

### BRIEF SUMMARY OF THE DRAWINGS

FIG. 1 shows the use of a blood glucose test strip being in accordance with the disclosure.

FIG. 2 is a block diagram of blood glucose meter hardware in accordance with the disclosure.

FIGS. 3A-3D are detailed views of a blood glucose meter in accordance with the disclosure.

FIG. 4 is an architecture diagram of a blood glucose meter in accordance with the disclosure.

FIG. 5 is a diagram illustrating a blood glucose measurement workflow in accordance with the disclosure.

FIG. 6 is a block diagram of a computing device in accordance with the disclosure.

### DETAILED DESCRIPTION

Disclosed is a system, method, and computer program product for improving the accuracy of blood glucose measurements. To obtain an accurate blood glucose measurement from a blood glucose test strip, it is necessary to determine the temperature of the enzymatic reaction site. Because a blood glucose meter may include heat sources, such as microprocessors, batteries, charging circuits, display devices, etc., that can influence temperature measurements near the test strip or reaction site, it is possible to improve the accuracy of blood glucose measurements by accounting for this influence in a temperature measurement taken near the test strip. To account for this influence, machine learning is used to generate a model that receives a plurality of inputs, including the ambient temperature near the test strip and the status or activity level of one or more heat sources in the vicinity of the test strip. Based on these inputs and a set of coefficients determined by training the model, the model outputs a predicted actual temperature value at the reaction site of the test strip, thereby allowing the blood glucose meter to more accurately calculate a blood glucose level in a blood sample.

In operation, in one embodiment, the user applies a sample of blood to a glucose test strip and inserts the test strip into the test strip interface of the glucometer. The glucometer measures the electrical current generated by the test strip at the test strip interface. The glucometer also obtains an ambient temperature reading from a first temperature sensor located near the test strip interface. The glucometer further obtains status information relating to any one or more of the other temperature sensors, hardware devices, and/or software applications discussed above. The glucometer provides the ambient temperature and the status information to the predictive thermal model, which then uses the status information to adjust the ambient temperature reading to a predicted actual temperature of the reaction site of the test strip. This predicted actual temperature and the measured electrical characteristics of the test strip are then provided to a blood glucose calculation module, which uses these values to calculate the amount of glucose in the sample of blood. The blood glucose calculation module then returns the blood glucose calculation to the glucometer software, which may then display the glucose calculation on the display for the user to see.

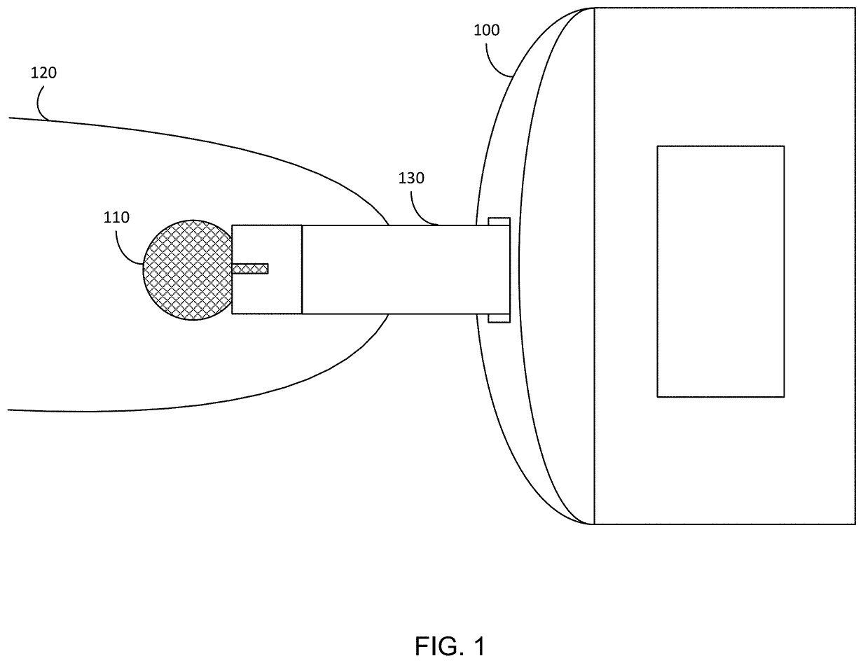

FIG. 1 illustrates a use case of a blood glucose meter (“glucometer”) 100. The user may draw a drop of blood 110 by pricking their finger 120 with a lancet or other sharp object (not shown). The blood drop 110 is applied to a test strip 130, which absorbs a portion or sample of the blood drop 110. The test strip 130 is inserted into the glucometer 100. The glucometer 100 analyzes the electric characteristics of the test strip 130 to calculate and display the glucose level in the blood sample.

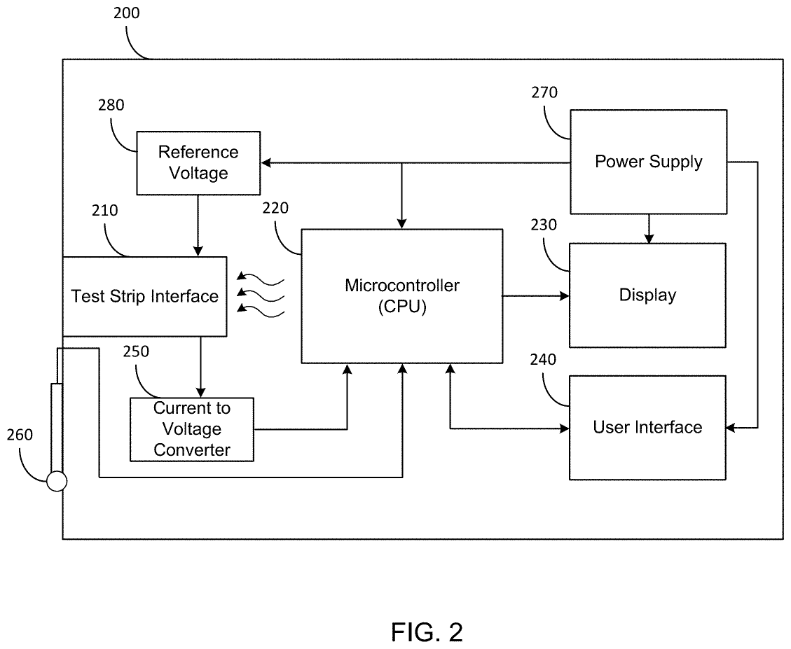

FIG. 2 is a block diagram showing the components of a glucometer in accordance with the disclosure. These components may be enclosed within or coupled to a portable, handheld housing (not shown). The components may be mounted on one or more printed circuit boards 200. The PCB 200 may also provide electrical coupling between the various components. The glucometer may include a test strip interface 210 configured to receive a blood glucose test strip, as discussed with respect to FIG. 1. An example glucometer in accordance with the disclosure is the Livongo Blood Glucose Monitoring System manufactured by LIVONGO, INC. The glucometer may also include a microcontroller or central processing unit (CPU) 220 that is coupled to a display 230, a user interface 240, the test strip interface 210 via a current to voltage converter (CVC) 250, a blood glucose (BG) thermistor 260, and a power supply 270.

Although not shown, power supply 270 may include a rechargeable battery as well as a charging port that can be coupled to a charging cable or other external power source to charge the battery and/or power the components of the glucometer.

Reference voltage 280 provides a predetermined electrical voltage that is applied to an electrical contact within the test strip interface 210. When a test strip is inserted into the test strip interface 210, an electrical current proportional the reference voltage 280 and the electrical resistance of the test strip is produced in the test strip. This current, which is indicative of the blood glucose level in the blood sample applied to the test strip, may be sensed by the CVC 250 and output to the CPU 220 in the form of a voltage signal.

CPU 220 executes software that controls or otherwise communicates with the other components of the glucometer. For example, CPU 220 may execute software that outputs information to be displayed by the display 230 and receives input from user interface 240, CVC 250, and BG thermistor 260.

BG thermistor 260 may be mounted near the test strip interface 210 to ensure any temperature readings from BG thermistor 260 reflect the ambient temperature around the test strip as nearly as possible. The CPU 220, display 230, power supply 270 and other components of glucometer 200 may generate heat (represented by wavy lines in FIG. 2). This heat may be transferred to the test strip interface 210 through convection, radiation, or conduction through the PCB 200. This heat may increase the temperature of the reaction site on the test strip beyond the ambient temperature sensed by the BG thermistor 260, resulting in a disparity between the actual reaction site temperature and the ambient temperature sensed by the BG thermistor 260.

Although not shown, additional temperature sensors may be disposed within the glucometer. For example, the glucometer may include one or more additional temperature sensors located on the PCB 200 and/or on embedded within the CPU 220 or other integrated circuits mounted on the PCB 200, such as a graphics processor, wireless network adapter, and/or charging circuit, etc. Any temperature sensor in communication with the CPU 220 can provide status information in the form of temperature measurements to the glucometer software running on CPU 220.

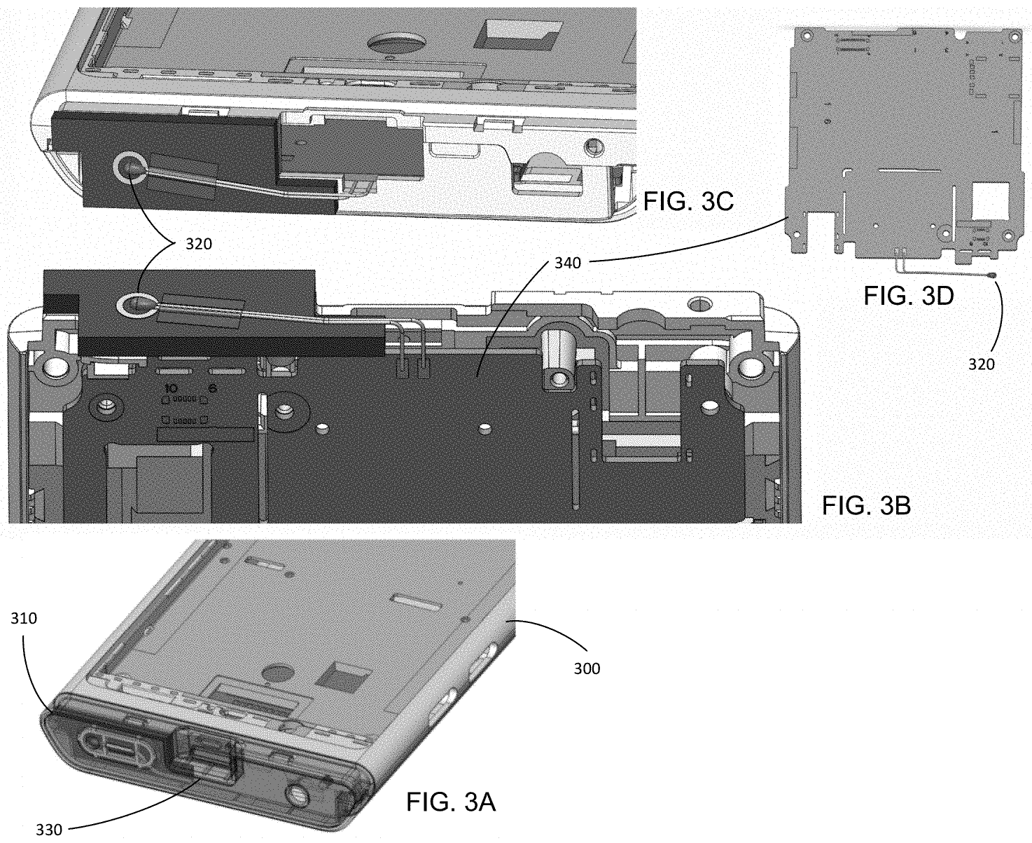

FIGS. 3A-D provide detailed views of a glucometer in accordance with the disclosure. The glucometer may include a housing 300 that has a cover 310 that encloses a BG thermistor 320 and has a recessed port 330 for receiving a test strip (not shown). The housing 300 also encloses a PCB 340 to which the BG thermistor 320 and other components are mounted and/or electrically coupled. As shown, the BG thermistor 320 may be located in proximity of the port 330 where the test strip is inserted. The location of the BG thermistor 320 may also be chosen to minimize the influence of heat generated within the glucometer so as to more accurately measure the ambient temperature around the glucometer. For example, as shown in FIG. 3A-D, the BG thermistor 320 is located proximate to the test strip port but nearer to the edge of the housing to reduce the influence of heat that may emanate from test strip port 330 and heat that may be conducted from the PCB 340 through the thermistor 330 leads. In addition, the material forming the leads connecting the BG thermistor 330 to the PCB 340 may be chosen to minimize heat transfer from the PCB 340. For example, the BG thermistor 330 leads may be composed of a nickel-copper alloy as opposed to pure copper.

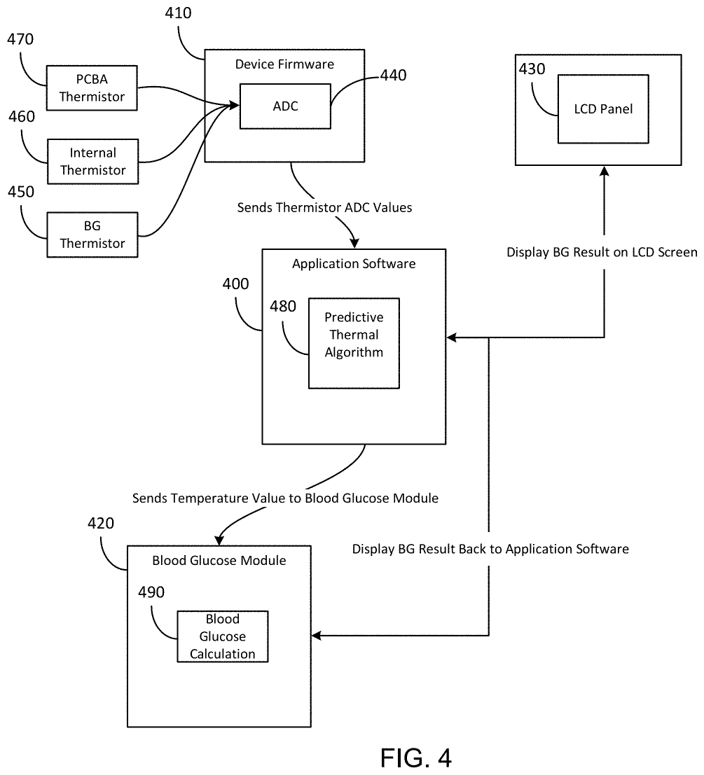

FIG. 4 is an architecture diagram of a glucometer in accordance with the disclosure. The architecture may include an application software 400, device firmware 410, blood glucose module 420, and a display 430. The application software 400 may execute on the glucometer CPU or microcontroller.

The firmware 410 may include an analog-to-digital converter (ADC) 440 that converts analog signals (e.g., voltage levels) from a thermistors 450, 460, and 470 into digital signals that can be interpreted as temperature readings by the application software 400. As discussed with respect to FIGS. 2 and 3A-D, the BG thermistor 450 may be located in proximity of the test strip port or interface on the glucometer. The internal thermistor 460 may be a through-hole mounted part located inside and near the edge of the glucometer housing that measures the temperature of the air inside the glucometer housing. The PCBA thermistor 470 may be a surface-mounted part located on the PCB within the glucometer housing but away from any hotspots such as the CPU or display to provide a more accurate reading of the glucometer PCB temperature.

The application software 400 may include a predictive thermal algorithm 480 that receives the temperature readings obtained from the thermistors 450, 460, 470 and other device status information and outputs a predicted temperature at the reaction site of a test strip inserted into the glucometer. This predicted temperature is output to blood glucose module 420 and used in a blood glucose calculation 490 to calculate the glucose level in the blood sample on the test strip. The calculated blood glucose level may be communicated back to the application software 400 which may then control the display 430 to display the calculated glucose level.

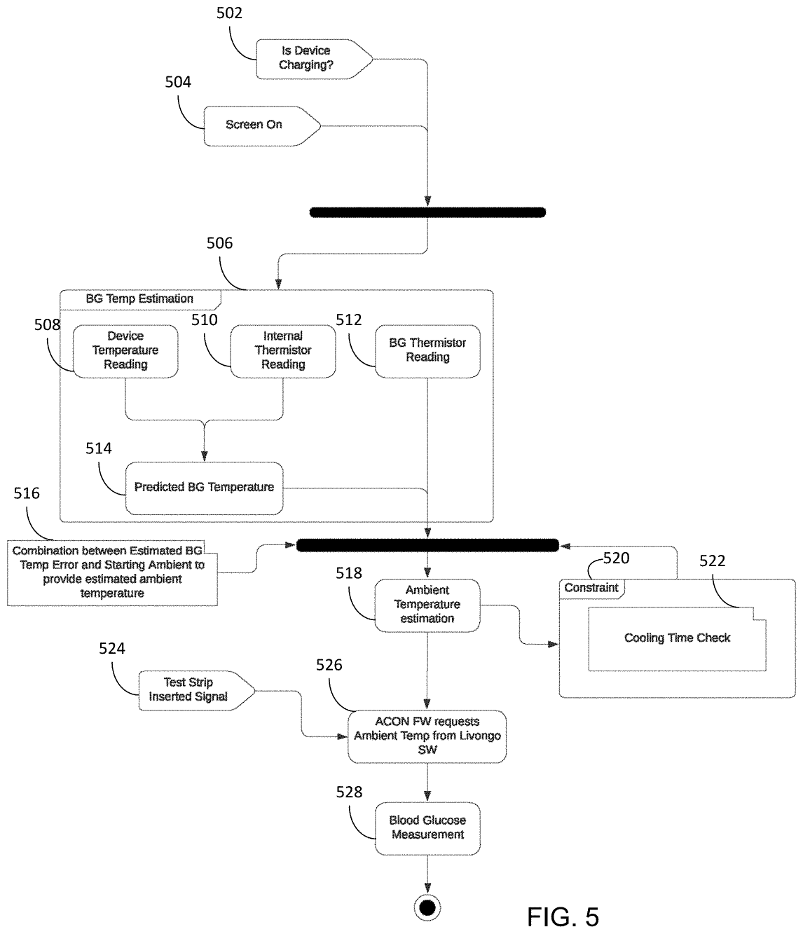

FIG. 5 is a diagram illustrating a blood glucose calculation workflow in a glucometer in accordance with the disclosure. At all times, device status information such as whether the device is charging 502 and whether the screen or display is active 504 is fed to the BG Temp Estimation block 506. At the BG Temp Estimation block 506, device status information 502, 504 is combined with instantaneous temperature readings from the various thermistors or temperature sensors disposed throughout the device, including the PCBA/Device Temperature Reading 508, Internal Thermistor Reading 510, and BG Thermistor Reading 512. These instantaneous temperature readings and status information are combined to calculate the Predicted BG Temperature 514, which represents a predicted error or delta between the instantaneous BG Thermistor reading and the predicted actual temperature of the reaction site of a test strip inserted into the glucometer. This predicted error value is combined with the instantaneous BG thermistor reading to generate an estimated ambient temperature at 516.

The estimated ambient temperature 518 may additionally be subject to one or more constraints 520 based on other device status information. For example, this may include a Cooling Time Check 522 that resets the BG Temp Estimation 506 to an initial state after a predetermined period of device inactivity after which any accumulated heat energy will have dissipated and the estimated ambient temperature 518 will be the same as the BR thermistor reading 512.

At 524, when a test strip is inserted into the glucometer, a test strip inserted signal triggers the glucometer firmware (“ACON FW”) to request the ambient temperature for the glucometer software. After the glucometer software responds to the glucometer firmware with the estimated ambient temperature from block 518, the firmware can complete the blood glucose measurement at 528 using the ambient temperature estimation.

The ambient temperature estimation can account for a variety of temperature sensor inputs and other device status information. For example, a battery charging circuit may control the interface between a rechargeable battery and an external energy source, such as a charging cable. The charging circuit is also in communication with the CPU and can provide status information to the glucometer software. Such status information may include the remaining charge in the battery, whether a charging cable is currently connected to the device, how long a charging cable has been connected to the device, and/or how recently a charging cable was connected or disconnected. If either or both of the charging circuit or the battery itself includes an internal temperature sensor, temperature readings from these sensors can also be provided to the glucometer software as status information.

The glucometer may also include a display device coupled to the CPU that provides status information to the glucometer software regarding the operational status of the display. For example, the glucometer software may track how long the display has been active, inactive, and/or how recently the display switched from active to inactive, or vice versa. The glucometer software may track other information relating to the display that may be indicative of the thermal characteristics of the display, such as backlighting levels and/or average luminosity of the display's pixels. If the display and/or its controller include one or more internal temperature sensors, temperature readings from these sensors may also be provided to the glucometer software as status information.

The glucometer software may also track status information relating to other devices, such as the activity of wireless networking circuits. For example, the glucometer software may track status information indicating whether and for how long a WIFI or cellular network interface in the glucometer has been active or inactive.

The glucometer software may additionally or alternatively track status information relating to certain software applications whose activity may influence the temperature readings from the test strip interface. For example, computationally intensive and/or bandwidth intensive applications running on the glucometer may produce elevated heat levels within the glucometer that can influence the actual temperature of the test strip. Applications such as these may include video streaming applications such as YOUTUBE and augmented reality or virtual reality applications that require intensive graphics rendering. The glucometer software may track status information indicative of whether, how long, and how recently applications such as these have been active on the glucometer.

Any or all of the temperature and/or status information discussed above may be input into a predictive thermal model associated with the glucometer. The predictive thermal model includes coefficients associated with each of the various inputs that represent the relative influence of each of input on the actual temperature at the reaction site on the test strip. These coefficients may be determined by testing the device in various use cases and using a machine learning algorithm to empirically characterize the effect of the various inputs on the actual temperature at the reaction site.

Any suitable machine learning or artificial intelligence model may be used to implement the predictive thermal algorithm. In addition, the thermal compensation may employ multi-regression model to estimate ambient temperature effects of the environment. For example, to estimate what the BG thermistor should be reading, the thermal compensation algorithm can use readings from the various thermistors combined with a multi-regression model to predict what the BG thermistor should be reading based on of previous data points. If the actual BG thermistor reading differs, it can be assumed that the difference between the estimated reading and actual reading is due to ambient temperature effects in the environment. Moreover, the algorithm may use previous device temperature and internal temperature to perform a multi-regression against previously measured BG thermistor temperatures. After this calculation is done, future BG thermistor readings can be approximated using the linear coefficients from the regression model.

The thermal compensation algorithm may additionally employ a dynamic regression model. A dynamic regression may be used to predict temperature values for use cases where the user is interacting with the device. These use cases can include activities such as normal BG checks, interacting with device for checking historical logs, on-boarding, and/or member support. Given this large variety in use case, a dynamic regression model may be valuable, as the user's heat generation may be heavily dependent on the use case and how strenuous that use case is on the device. By way of example, to perform a dynamic regression analysis, the model may employ a block array of 40 samples. To achieve this, the predictive thermal algorithm may collect 40 samples over a predetermined time period, e.g., 10 seconds, on which to perform the regression. The predetermined time period may be chosen based on empirical testing of the device's thermal characteristics. For example, testing may be conducted on the device to establish a maximum expected rate of change of device temperature over time. If, for example, this testing establishes that the device only increases at a maximum of 1° C. per minute, a dynamic regression time interval of 10 seconds will be sufficient to capture any inflections of the temperature gradient through this specified time cycle. In such case, while the device is in active use, this re-calculation would be performed every 10 seconds. After the device becomes idle, the recalculation is done less frequently (e.g., every 15 minutes), until the cooling off timeout is reached, after which the thermal compensation algorithm is suspended. When the device becomes active again, the thermal compensation algorithm is reset to the initial state and the cycle repeats.

Although the above-described embodiments pertain to improving accuracy in blood glucose measurements, it is contemplated that these systems and methods could be applied in any context to improve the accuracy of temperature measurements where such measurements may be influenced by the thermal characteristics of proximal heat sources.

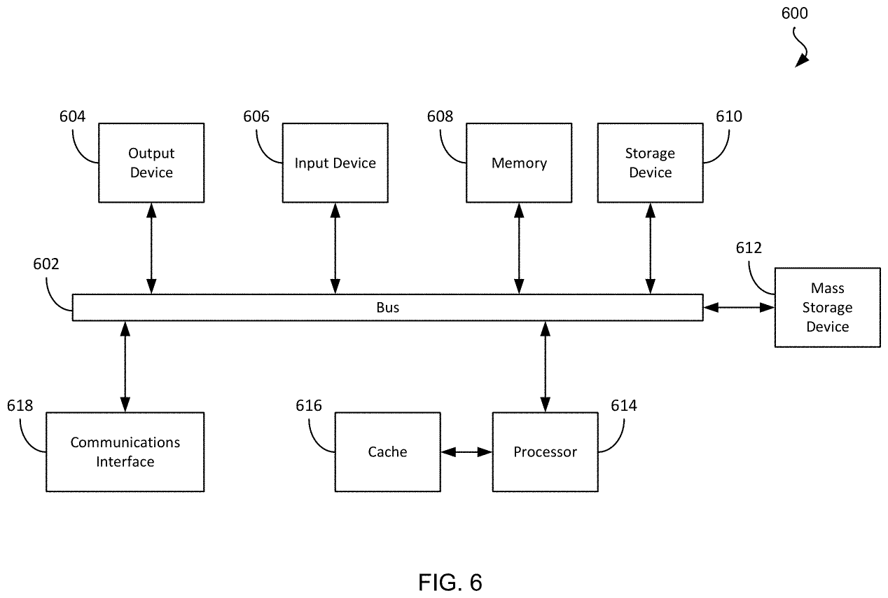

FIG. 6 depicts an example computing system 600 that may implement various systems and methods discussed herein. The computer system 600 includes one or more computing components in communication via a bus 602. In one implementation, the computing system 600 includes one or more processors 614. The processor 614 can include one or more internal levels of cache 616 and a bus controller or bus interface unit to direct interaction with the bus 602. Memory 608 may include one or more memory cards and control circuits (not depicted), or other forms of removable memory, and may store various software applications including computer executable instructions, that when run on the processor 614, implement the methods and systems set out herein. Other forms of memory, such as a mass storage device 612, may also be included and accessible, by the processor (or processors) 614 via the bus 602.

The computer system 600 may further include a communications interface 618 by way of which the computer system 600 can connect to networks and receive data useful in executing the methods and system set out herein as well as transmitting information to other devices. The computer system 600 may include an output device 604 by which information can be displayed. The computer system 600 can also include an input device 606 by which information is input. Input device 606 can be a scanner, keyboard, and/or other input devices as will be apparent to a person of ordinary skill in the art.

The system set forth in FIG. 6 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

We claim:

1. A blood glucose meter comprising:
a test strip interface configured to be electrically coupled to a test strip that contains a blood sample;
a first temperature sensor located a first distance from the test strip interface;
a user interface; and
a first processor coupled to the test strip interface, the first temperature sensor, and the user interface, wherein the processor is configured to calculate a glucose value associated with the blood sample based on a plurality of inputs, the inputs including an electrical characteristic of the test strip, a first temperature value received from the first temperature sensor, and a device status indicator.

2. The blood glucose meter of claim 1, further comprising a second temperature sensor located a second distance from the test strip interface, and wherein the device status indicator is a second temperature value received from the second temperature sensor.

3. The blood glucose meter of claim 1, further comprising a battery coupled to a charging circuit that is coupled to the processor, and wherein the device status indicator is a charging status.

4. The blood glucose meter of claim 1, wherein the device status indicator is a utilization level of the first processor.

5. The blood glucose meter of claim 1, further comprising a second processor, and wherein the device status indicator is a utilization level of the second processor.

6. The blood glucose meter of claim 1, wherein the device status indicator includes an indication of a status of a first software application.

7. The blood glucose meter of claim 1, further comprising a battery and the device status indicator includes an indication of the status of the battery.

8. The blood glucose meter of claim 7, wherein the indication of the status of the battery includes a charging status.

9. The blood glucose meter of claim 1, wherein the device status indicator includes an indication of the status of the user interface.

10. The blood glucose meter of claim 1, wherein each of the plurality of inputs is weighted in accordance with a trained artificial intelligence (AI) model.

11. A method for estimating reaction site temperature in a blood glucose meter, the method comprising:
obtaining, at a first processor, a measurement of an electrical characteristic of a blood glucose test strip from test strip interface of a blood glucose meter;
obtaining, at the first processor, a first temperature value from a first temperature sensor located a first distance from the test strip interface;
obtaining, at the first processor, at least one device status indicator;
calculating a blood glucose measurement based on a plurality of inputs including the measured electrical characteristic, the first temperature value, and the at least one status indicator, wherein each of the plurality of inputs is weighted in accordance with a model; and,
displaying the calculated blood glucose measurement on a user interface coupled to the first processor.

12. The method of claim 11, wherein the at least one status indicator is a second temperature sensor value obtained from a second temperature sensor located a second distance from the test strip interface.

13. The method of claim 11, wherein the at least one status indicator is a charging status obtained from a battery charging circuit coupled to the processor.

14. The method of claim 11, wherein the at least one status indicator is a utilization level of the first processor.

15. The method of claim 11, wherein the at least one status indicator is a utilization level of a second processor coupled to the first processor.

16. The method of claim 11, wherein the at least one status indicator is a status of a first software application.

17. The method of claim 11, wherein the model comprises a trained artificial intelligence (AI) model.

18. A non-transitory computer-readable medium storing program code that, when executed by a processor, cause the processor to perform a method for estimating reaction site temperature in a blood glucose meter, the method comprising:
obtaining a measurement of an electrical characteristic of a blood glucose test strip from test strip interface of a blood glucose meter;
obtaining a first temperature value from a first temperature sensor located a first distance from the test strip interface;
obtaining at least one device status indicator;
calculating a blood glucose measurement based on a plurality of inputs including the measured electrical characteristic, the first temperature value, and the at least one status indicator, wherein each of the plurality of inputs is weighted in accordance with a model; and,
displaying the calculated blood glucose measurement on a user interface.

19. The method of claim 11, wherein the model comprises a trained artificial intelligence (AI) model.
